SEQUENCE LISTING (30 sequences)

SEQ ID NO: 1 - protein, 22 aa, Artificial Sequence
  Matrix-binding domain
KKKRKGKGLGKKRDPCLKKYKG

SEQ ID NO: 2 - protein, 22 aa, Artificial Sequence
  Matrix-binding domain
MKRKKKGKGLGKKRDPCLRKYK

SEQ ID NO: 3 - protein, 21 aa, Artificial Sequence
  Matrix-binding domain
KRKKKGKGLGKKRDPCLRKYK

SEQ ID NO: 4 - protein, 22 aa, Artificial Sequence
  Matrix-binding domain
MKRKKKGKGLGKKRDPSLRKYK

SEQ ID NO: 5 - protein, 22 aa, Artificial Sequence
  Matrix-binding domain
MKRKKKGKGLGKKRDPRLRKYK

SEQ ID NO: 6 - protein, 22 aa, Artificial Sequence
  Matrix-binding domain
MKRKKKGKGLGKKRDPKLRKYK

SEQ ID NO: 7 - protein, 21 aa, Artificial Sequence
  Matrix-binding domain
KRKKKGKGLGKKRDPSLRKYK

SEQ ID NO: 8 - protein, 21 aa, Artificial Sequence
  Matrix-binding domain
KRKKKGKGLGKKRDPRLRKYK

SEQ ID NO: 9 - protein, 21 aa, Artificial Sequence
  Matrix-binding domain
KRKKKGKGLGKKRDPKLRKYK

SEQ ID NO: 10 - protein, 17 aa, Artificial Sequence
  Matrix-binding domain
RIQNLLKITNLRIKFVK

SEQ ID NO: 11 - protein, 20 aa, Artificial Sequence
  Matrix-binding domain
PYVVLPRPVCFEKGMNYTVR

SEQ ID NO: 12 - protein, 22 aa, Artificial Sequence
  Matrix-binding domain
KQNCLSSRASFRGCVRNLRLSR

SEQ ID NO: 13 - protein, 25 aa, Artificial Sequence
  Matrix-binding domain
KDGRKICLDLQAPLYKKIIKKLLES

SEQ ID NO: 14 - protein, 22 aa, Artificial Sequence
  Matrix-binding domain
CKNGGFFLRIAPDGRVDGVREK

SEQ ID NO: 15 - protein, 31 aa, Artificial Sequence
  Matrix-binding domain
YSSWYVALKRTGQYKLGPKTGPGQKAILFLP

SEQ ID NO: 16 - protein, 27 aa, Artificial Sequence
  Matrix-binding domain
AKLNCRLYRKANKSSKLVSANRLFGDK

SEQ ID NO: 17 - protein, 23 aa, Artificial Sequence
  Matrix-binding domain
LRKLRKRLLRDADDLQKRLAVYQ

SEQ ID NO: 18 - protein, 42 aa, Artificial Sequence
  Matrix-binding domain
PLQERAQAAWQERLRARMEEMGSRTRDRLDEVKEQVAERAKL

SEQ ID NO: 19 - protein, 10 aa, Artificial Sequence
  Matrix-binding domain
KGKMHKTCYY

SEQ ID NO: 20 - protein, 10 aa, Artificial Sequence
  Matrix-binding domain
MGKMHKTCYN

SEQ ID NO: 21 - protein, 28 aa, Artificial Sequence
  Matrix-binding domain
PPTIIWKHKGRDVILKKDVRFIVLSNNY

SEQ ID NO: 22 - protein, 22 aa, Artificial Sequence
  Matrix-binding domain
KKHEAKNWFVGLKKNGSCKRGP

SEQ ID NO: 23 - protein, 32 aa, Artificial Sequence
  Matrix-binding domain
KGGRGTPGKPGPRGQRGPTGRGERGPRGITGK

SEQ ID NO: 24 - protein, 12 aa, Artificial Sequence
  Matrix-binding domain
GEFYDLRLKGDK

SEQ ID NO: 25 - protein, 15 aa, Artificial Sequence
  Matrix-binding domain
HRHHPREMKKRVEDL

SEQ ID NO: 26 - protein, 14 aa, Artificial Sequence
  Matrix-binding domain
EKTLRKWLKMFKKR

SEQ ID NO: 27 - protein, 19 aa, Artificial Sequence
  Matrix-binding domain
AEAAARAAARRAARRAAAR

SEQ ID NO: 28 - protein, 178 aa, Artificial Sequence
  Matrix-binding domain-IL-1RA fusion protein
MKRKKKGKGLGKKRDPCLRKYKGGGSRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 29 - protein, 178 aa, Artificial Sequence
  Matrix-binding domain-IL-1RA fusion protein
MKRKKKGKGLGKKRDPRLRKYKGGGSRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 30 - protein, 98 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSA

(19) United States
(12) Patent Application Publication
(10) Pub. No.: US 2019/0016754 A1
(43) Pub. Date: Jan. 17, 2019

(54) METHODS OF PRODUCING AND PURIFYING MATRIX-BINDING FUSION PROTEINS BY ION-EXCHANGE CHROMATOGRAPHY

(71) Applicant: ProteoThera, Inc., Cambridge, MA (US)

(72) Inventor: Parth PATWARI, Cambridge, MA (US)

(21) Appl. No.: 15/774,424

(22) PCT Filed: Nov. 10, 2016
(86) PCT No.: PCT/US16/61284

§ 371 Date: May 8, 2018

Related U.S. Application Data
(60) Provisional application No. 62/253,277, filed on Nov. 10, 2015.

(51) Int. Cl. C07K 1/22; C07K 16/22; C07K 14/575; C07K 14/48; C12N 15/62; C07K 16/06; C07K 14/54

(52) U.S. Cl. CPC C07K 1/22; C07K 16/22; C07K 14/575; C07K 14/48; C12N 15/62; C07K 2319/20; C07K 14/5406; C07K 14/5412; C07K 14/5428; C07K 14/5431; C07K 16/065

(57) ABSTRACT

The invention provides methods of producing and purifying fusion proteins containing a domain capable of binding one or more extracellular matrix components, such as heparin and chondroitin sulfate, by cation-exchange chromatography.

### FIELD OF THE INVENTION

The invention relates to the fields of analytical chemistry and molecular biology, as the methods described herein can be used to separate proteins containing particular physicochemical characteristics from a complex mixture of various biological molecules.

### BACKGROUND OF THE INVENTION

Affinity tags are commonly used to allow purification of proteins after recombinant expression for research applications. However, these affinity methods are often not scalable for commercial production (Fong et al. Trends Biotechnol. 28:272-279 (2010); Nilsson et al. Protein Expr. Purif. 11:1-16 (2007); and Terpe et al. Appl. Microbiol. Biotechnol. 60:523-533 (2003); the disclosures of each of which are incorporated herein by reference). Affinity tags typically require special affinity chromatography steps for purification. In addition, for production of therapeutic proteins, standard affinity tags do not contribute to therapeutic efficacy and must be removed from the protein by cleavage to prevent immunogenic responses to the protein. This requires use of a relatively expensive endopeptidase to cleave the tag from the protein, and an additional purification step to remove the endopeptidase and the cleaved tag. In addition, even for research-scale applications, cleaved tags can be undesirable since they typically leave one or two unwanted residues on the cleaved protein that can affect the structure or activity of the protein.

Protein fusions with short oligomers of arginine were originally described in the 1980s for facilitating purification of proteins via inexpensive cation-exchange chromatography (Sassenfeld et al. Trends Biotechnol. 8:88-93 (2003), the disclosure of which is incorporated herein by reference). However, polyarginine tags are not commonly used due to problems with incomplete cleavage of the tag, the effects of the polyarginine tag on protein stability and internalization, and potential for immunogenicity since it is not an endogenously occurring sequence (Fuchs et al. Protein Sci. Publ. Protein Soc. 14:1538-1544 (2005) and Terpe et al. Appl. Microbiol. Biotechnol. 60:523-533 (2003), the disclosures of each of which are incorporated herein by reference).

### SUMMARY OF THE INVENTION

In a first aspect, the invention relates to a method of purifying a fusion protein containing a matrix-binding domain. The method includes contacting a mixture of polypeptides containing the fusion protein with a substance that has one or more negatively-charged agents so that the matrix-binding domain of the fusion protein specifically binds the one or more negatively-charged agents from the mixture, thereby producing a mixture that is enriched with the fusion protein.

In some embodiments, the substance that has one or more negatively-charged agents is contained within a column, which can optionally be in fluid connection with one or more pumps.

In some embodiments, the matrix-binding domain is capable of specifically binding a glycosaminoglycan selected from the group consisting of heparin, heparan sulfate, chondroitin sulfate, dermatan sulfate, and hyaluronic acid. In some embodiments, the matrix-binding domain has at least 85% sequence identity (e.g., at least 85%, 90%, 95%, 97%, 98%, 99%, or more, sequence identity) to the amino acid sequence of any one of SEQ ID NOs: 1-27.

In some embodiments, the fusion protein comprises a therapeutic polypeptide. For instance, the therapeutic polypeptide may be selected from the group consisting of growth and differentiation factor 11 (GDF11), stromal cell-derived factor 1 (SDF-1), growth and differentiation factor 8 (GDF8), insulin-like growth factor 1 (IGF-1), parathyroid hormone (PTH), parathyroid hormone related peptide (PTHrP), interleukin 1 receptor antagonist (IL-1RA), fibroblast growth factor 9 (FGF-9), fibroblast growth factor 18 (FGF-18), high-mobility group protein 2 (HMG-2), hepatocyte growth factor, transforming growth factor β (TGFβ), transforming growth factor β3 (TGFβ3), bone morphogenetic protein 2 (BMP2), bone morphogenetic protein 7 (BMP7), angiopoietin-like 3 (ANGPTL3), and somatostatin (SST).

In some embodiments, the therapeutic polypeptide includes an antibody or an antigen-binding fragment thereof. For example, the antibody may be selected from the group consisting of infliximab, adalimumab, etanercept, and an anti-nerve growth factor antibody.

In some embodiments, the therapeutic polypeptide is a neurotrophin, such as a neurotrophin selected from the group consisting of nerve growth factor (NGF), brain-derived neurotrophic factor (BDNF), neurotrophin-3 (NT-3), and neurotrophin-4 (NT-4).

In some embodiments, the therapeutic polypeptide is a neurotrophic factor. For instance, neurotrophic factor may be selected from the group consisting of glial cell line-derived neurotrophic factor (GDNF), neurturin (NRTN), artemin (ARTN), persephin (PSPN), ciliary neurotrophic factor (CNTF), mesencephalic astrocyte-derived neurotrophic factor (MANF), and conserved dopamine neurotrophic factor (CDNF).

In some embodiments, the therapeutic polypeptide is a cytokine, such as a cytokine selected from the group consisting of interleukin-4, interleukin-6, interleukin-10, interleukin-11, interleukin-27, leukemia inhibitory factor, cardiotrophin 1, neuropoietin, and cardiotrophin-like cytokine.

In some embodiments, the therapeutic polypeptide is a neuroprotection agent, such as a neuroprotection agent selected from the group consisting of Neuregulin-1 and vascular endothelial growth factor (VEGF).

In some embodiments, the fusion protein contains a linker. For instance, the linker may include a peptide linker that has one or more amino acids, such as D- or L-amino acids and non-naturally occurring amino acids, or combinations thereof, or a non-peptide linker. In some embodiments, the linker is cleavable, e.g., by a process selected from the group consisting of enzymatic hydrolysis, photolysis, hydrolysis under acidic conditions, hydrolysis under basic conditions, oxidation, disulfide reduction, nucleophilic cleavage, and organometallic cleavage. The linker may include a polypeptide of the formula [(Gly)ₐ(Ser)_{b}]_{c}, wherein a, b, and c are independently integers from 0 to 20. In some embodiments, b is 0. In some embodiments, a is 3. In some embodiments, a is 3 or 4 and b is 1. In some embodiments, a is 3 or 4, b is 1, and c is an integer from 1 to 6 (e.g., 1, 2, 3, 4, 5, or 6).

In some embodiments, the fusion protein is isolated from a cell, such as a eukaryotic cell (e.g., a mammalian cell, such as a human cell) or a prokaryotic cell (e.g., a bacterial cell, such as an E. coli cell). In some embodiments, the fusion protein is produced by treating the E. coli cell with isopropyl-ρ-D-thiogalactoside (IPTG).

In some embodiments, the method includes contacting the one or more negatively-charged agents with a solution including a dissolved cation. This contacting can cause the fusion protein to dissociate from the substance including one or more negatively-charged agents. In some embodiments, the dissolved cation is selected from the group consisting of lithium (Li⁺), sodium (Na⁺), potassium (K⁺), ammonium (NH₄⁻), magnesium (Mg²⁺), calcium (Ca²⁺), and zinc (Zn⁺).

In some embodiments, the method includes contacting the one or more negatively-charged agents with a first solution containing the dissolved cation, and subsequently contacting the one or more negatively-charged agents with a second solution containing the dissolved cation. In this step, the concentration of the dissolved cation in the second solution is greater than the concentration of the dissolved cation in the first solution. The method may include subsequently contacting the one or more negatively-charged agents with a third solution containing the dissolved cation, wherein the concentration of the dissolved cation in the third solution is greater than the concentration of the dissolved cation in the first solution and the second solution. For instance, the concentration of the dissolved cation in the first solution may be from about 1 to about 100 mM (e.g., about 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM). In some embodiments, the concentration of the dissolved cation in the first solution is about 50 mM. In some embodiments, the concentration of the dissolved cation in the second solution is from about 500 mM to about 1.5 M (e.g., about 500 mM, 600 mM, 700 mM, 800 mM, 900 mM, 1.0 M, 1.2 M, 1.3 M, 1.4 M, or 1.5 M). In some embodiments, the concentration of the dissolved cation in the second solution is about 1 M. In some embodiments, the concentration of the dissolved cation in the third solution is from about 1.6 M to about 2.5 M (e.g., about 1.6 M, 1.7 M, 1.8 M, 1.9 M, 2.0 M, 2.1 M, 2.2 M, 2.3 M, 2.4 M, or 2.5 M). In some embodiments, the concentration of the dissolved cation in the second solution is about 2 M.

In some embodiments, the first, second, and third solutions flow through the substance including one or more negatively-charged agents at a rate of from about 1 mL/minute to about 3 mL/minute (e.g., about 1.0 mL/minute, 1.1 mL/minute, 1.2 mL/minute, 1.3 mL/minute, 1.4 mL/minute, 1.5 mL/minute, 1.6 mL/minute, 1.7 mL/minute, 1.8 mL/minute, 1.9 mL/minute, 2.0 mL/minute, 2.1 mL/minute, 2.2 mL/minute, 2.3 mL/minute, 2.4 mL/minute, 2.5 mL/minute, 2.6 mL/minute, 2.7 mL/minute, 2.8 mL/minute, 2.9 mL/minute, or 3.0 mL/minute). In some embodiments, the first, second, and third solutions flow through the substance including one or more negatively-charged agents at a rate of about 1.25 mL/minute.

In some embodiments, the one or more negatively-charged agents are selected from the group consisting of methanesulfonic acid, ethanesulfonic acid, propanesulfonic acid, benzenesulfonic acid, and acetic acid. The one or more negatively-charged agents may be covalently bound to the substance. In some embodiments, the substance is a polysaccharide, such as agarose. In some embodiments, the substance is polystyrene. In some embodiments, the substance contains one or more hydrophobic molecules.

In some embodiments, the methods of the invention include:
a) (i) contacting the mixture that is enriched with the fusion protein with a material including a plurality of particles; and
   (ii) separating polypeptides that flow through the material from polypeptides that remain within the material, or
b) (i) contacting the mixture that is enriched with the fusion protein with a material including one or more hydrophobic molecules; and
   (ii) separating polypeptides that bind the one or more hydrophobic molecules from the mixture that is enriched with the fusion protein.

In some embodiments, the methods of the invention include, in either order:
a) (i) contacting the mixture that is enriched with the fusion protein with a material including a plurality of particles; and
   (ii) separating polypeptides that flow through the material from polypeptides that remain within the material, and
b) (i) contacting the mixture that is enriched with the fusion protein with a material including one or more hydrophobic molecules; and
   (ii) separating polypeptides that bind the one or more hydrophobic molecules from the mixture that is enriched with the fusion protein.

In some embodiments, the average diameter of the plurality of particles is from about 1 μm to about 100 μm (e.g., about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, or 100 μm). In some embodiments, the average diameter of the plurality of particles is from about 10 μm to about 50 μm (e.g., about 10 μm, 15 μm, 20 μm, 25 μm, 30 μm, 35 μm, or 40 μm). In some embodiments, the average diameter of the plurality of particles is about 34 μm.

In some embodiments, the fusion protein contains the amino acid sequence of SEQ ID NO: 28 or SEQ ID NO: 29, or a variant thereof, such as a variant having at least 85% sequence identity (e.g., at least 85%, 90%, 95%, 97%, 98%, 99%, or more, sequence identity) to the amino acid sequence of SEQ ID NO: 28 or SEQ ID NO: 29. In some embodiments, the fusion protein contains a variant of SEQ ID NO: 28 or SEQ ID NO: 29, such as a variant having at least 85% sequence identity (e.g., at least 85%, 90%, 95%, 97%, 98%, 99%, or more, sequence identity) to the amino acid sequence of SEQ ID NO: 28 or SEQ ID NO: 29, and that binds a glycosaminoglycan that is expressed in the extracellular matrix of a tissue, such as chondroitin sulfate, heparan sulfate, dermatan sulfate, and/or hyaluronic acid. In some embodiments, the fusion protein consists of SEQ ID NO: 28 or 29.

### Definitions

As used herein, the term “about” refers to a value that is within 10% above or below the value being described.

As used herein, the term “extracellular matrix” refers to the endogenous collection of collagens, elastins, laminins, glycosaminoglycans, proteoglycans, antimicrobials, chemoattractants, cytokines, growth factors, and other molecules located exterior to the cell membrane.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein, for instance, with or without a linker between the N- and C-terminal portions of the protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another protein. Examples of linkers that can be used for the formation of a fusion protein include peptide-containing linkers, such as those that contain naturally occurring or non-naturally occurring amino acids, as well as small molecule linkers. Exemplary linkers are described, e.g., in WO 2014/004465, the disclosure of which is incorporated herein by reference. In certain cases, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

As used herein, the term “matrix-binding domain” refers to a molecule, such as a polypeptide, that is capable of specifically binding a glycosaminoglycan that is expressed in the extracellular matrix of a tissue. Exemplary glycosaminoglycans expressed in the extracellular matrix include, without limitation, chondroitin sulfate, heparan sulfate, dermatan sulfate, and hyaluronic acid.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence (e.g., 60%, 70%, 80%, 90%, or 100% sequence identity). The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein with a K_{D} of less than 500 nM. For example, a ligand that specifically binds to a protein will bind to the protein with a K_{D} of up to 500 nM (e.g., between 1 pM and 500 nM). A ligand that does not exhibit specific binding to a protein or a domain thereof will exhibit a K_{D} of greater than 500 nM (e.g., greater than 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; the disclosure of which is incorporated herein by reference. Expression vectors may contain a polynucleotide sequence as well as, e.g., additional sequence elements used for the expression of proteins and/or the integration of these polynucleotide sequences into the genome of a mammalian cell. Certain vectors that can be used for the recombinant expression of proteins include plasmids that contain regulatory sequences, such as promoter and enhancer regions, which direct gene transcription. Other useful vectors for recombinant protein expression contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of the invention may also contain a polynucleotide encoding a marker for selection of cells that contain such a vector. Examples of a suitable marker include genes that encode resistance to antibiotics, such as ampicillin, chloramphenicol, kanamycin, or nourseothricin.

### BRIEF DESCRIPTION OF THE FIGURES

FIG. 1 is a graph showing the distribution of MB-IL1RA protein concentrations of fractions eluted from a cation-exchange chromatography column.

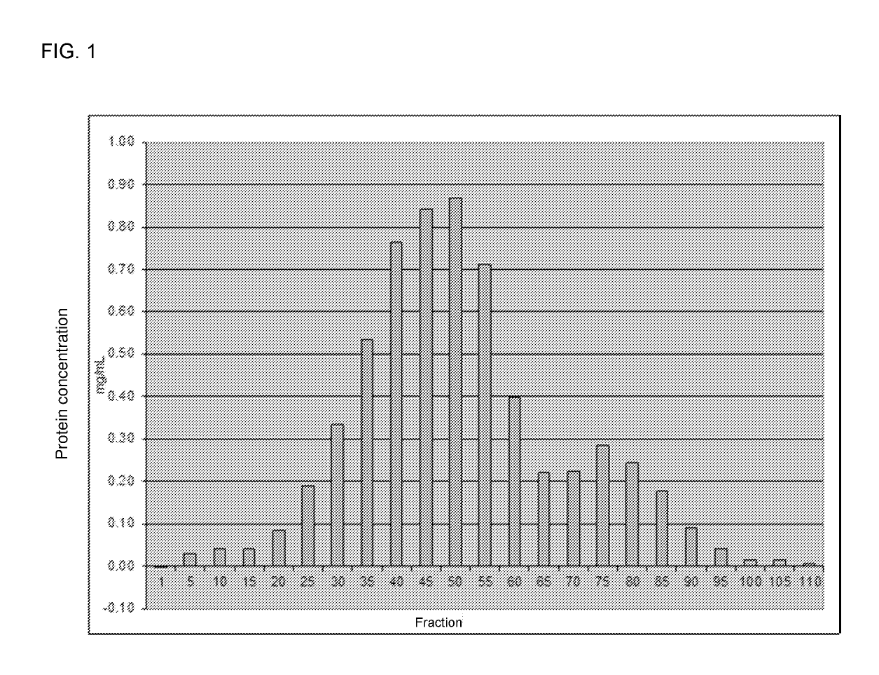

FIG. 2 is an image showing gel electrophoresis analysis of fractions obtained from the purification of MB-IL1RA by cation-exchange chromatography. The image shows SDS-PAGE analysis of cell lysates, cell pellet, and fractions from the cation-exchange chromatography column including the flow-through (FT2, FT10, and FT18), wash (W1), and elution with a NaCl gradient (fractions 20, 35, 50, 60, 68, 75, and 90).

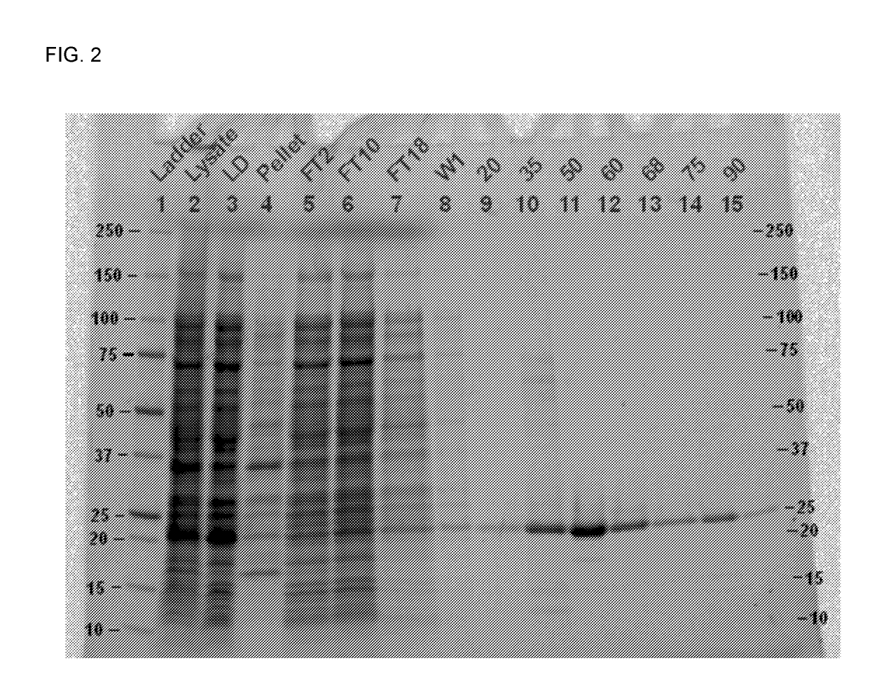

FIG. 3 is a fast protein liquid chromatography (FPLC) chromatogram showing the elution profile of MB-IL1RA from a Superdex 75 26/60 size-exclusion column following cation-exchange chromatography.

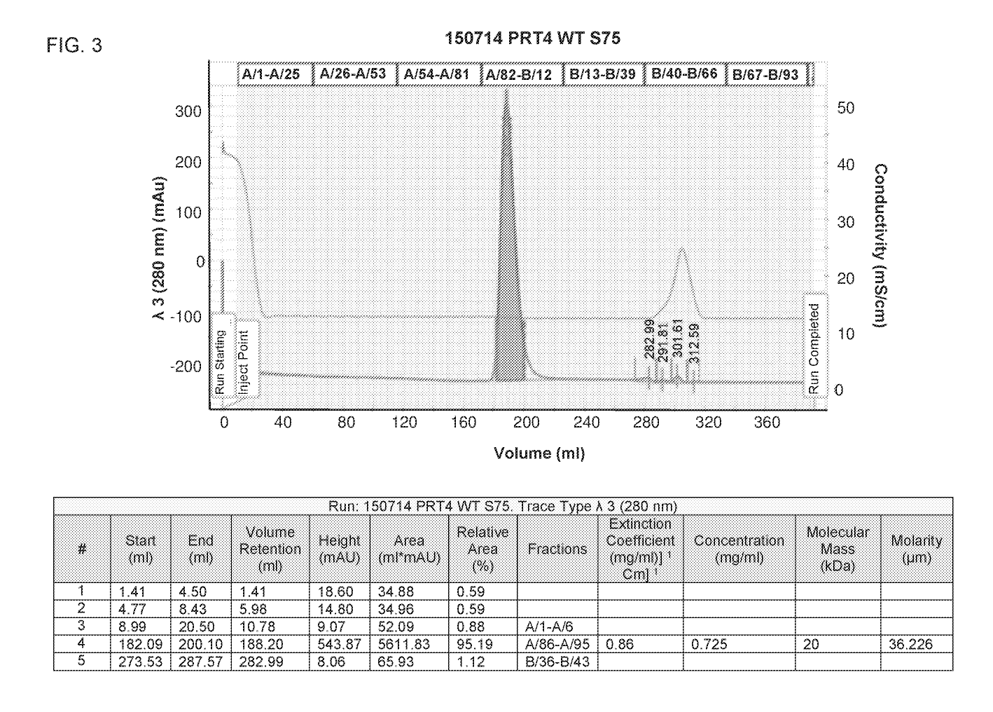

FIG. 4 is an image showing the SDS-PAGE analysis of pooled fractions eluted from the Superdex 75 26/60 size-exclusion chromatography column described in FIG. 3. Lanes 3-6 were loaded with 0.1, 0.5, 1, and 5 μg of purified MB-IL1RA protein respectively; lanes 7-9 were loaded with 0.1, 0.5, and 1 μg of bovine serum albumin (BSA) respectively, as a control.

FIG. 5 is a chromatogram showing the purification of MB-IL1RA by cation-exchange chromatography. The method utilized a SP Sepharose FF column (GE Healthcare) and a gradient elution profile of 50 mM NaCl to 1 M NaCl in a 50 mM sodium borate buffer at pH 9.6. The UV trace shown was recorded at 276 nm.

FIG. 6 is a chromatogram showing the purity of the MB-IL1RA sample obtained from the cation-exchange chromatography experiment described in FIG. 5.

FIG. 7 is a graph showing the dose-dependent inhibition of IL-1 activity induced by MB-IL1RA as purified by cation-exchange chromatography. The MB-IL1RA fusion protein exhibited a half-maximal inhibitory concentration (IC₅₀) of 281 pM. For comparison, the graph also shows the dose-dependent inhibition of IL-1 activity induced by anakinra (untagged pharmaceutical-grade Met-IL1RA, Sobi, Inc., Waltham, Mass.).

### DETAILED DESCRIPTION

I have discovered that fusion proteins containing an extracellular matrix-binding domain (e.g., a heparin-binding domain) can be purified effectively using cation-exchange chromatography and/or mixed-mode chromatography in which one mode of separation is through electrostatic interactions with the column's stationary phase. In particular, the yield and purity of recombinantly expressed fusion proteins containing a peptide domain that binds to charged glycosaminoglycans can be greatly improved when purified in this manner. The peptide domain may be encoded in an expression vector that expresses the peptide domain and protein together as a fusion protein. The fusion protein may include a cleavable linker that allows removal of the peptide domain after purification. Alternatively, the peptide domain is not cleaved and remains as part of the therapeutic protein.

We describe here a process for improving yield and purity of a recombinant protein of interest, e.g., protein “X”) expressed in prokaryotic or eukaryotic organisms using a matrix-binding domain (MB) by expressing a protein comprising MB-L-X or X-L-MB, where L is an optional linker. L may be cleavable, allowing production of protein, X, after purification and cleavage of the MB-L sequence. L may be absent or noncleavable, where the modified MB-L-X protein retains therapeutic activity.

To reduce immunogenicity, the matrix-binding domain can be derived from an endogenous heparin-binding domain of a human protein. These isolated heparin-binding domains can be found, or optimized with mutations, to bind to different extracellular-matrix-associated sulfated sugars or aminosugars such as chondroitin sulfate, heparan sulfate, dermatan sulfate, and hyaluronic acid (Miller et al. Arthritis Rheum. 62:3686-3694 (2010)).

Such a process can be used to improve yield and purity of a recombinant protein. As one example, we have prepared a fusion protein that contains IL-1RA, expressed in E. coli, as described in the examples below using a peptide sequence that binds to both heparan sulfate and chondroitin sulfate. Using a matrix-binding fusion protein, the protein is readily purified by cation-exchange chromatography, eluting at a high molarity of salt. Following cation-exchange chromatography, the matrix-binding fusion protein may be further purified by mixed-mode hydrophobic interaction chromatography.

### Purification Process

The methods of the invention can be used to purify fusion proteins containing a matrix-binding domain (e.g., a peptide that binds heparin, heparan sulfate, chondroitin sulfate, dermatan sulfate, or hyaluronic acid, such as a peptide that has at least 85% sequence identity (e.g., 85%, 90%, 91%, 92%, 93%, 94%, 95%, 96%, 97%, 98%, 99%, or 100% sequence identity) to the amino acid sequence of any one of SEQ ID NOs: 1-27) conjugated to a second molecule, such as a therapeutic polypeptide, optionally via a linker (e.g., a peptidic or small molecule linker known in the art or described herein). Fusion proteins of this structure can be prepared, e.g., using cell-based protein expression techniques known in the art, such as by recombinantly expressing a polynucleotide that encodes the fusion protein in a host cell (e.g., a bacterial cell) and that is under the control of an inducible regulatory sequence, such as a T7 promoter that drives gene expression by binding T7 RNA polymerase. The components of the cell culture system that contain the fusion protein can be isolated, e.g., by lysing the cells using standard techniques, such as by sonication followed by centrifugation, in order to separate aqueous fractions from membrane-soluble components of the system, which form pellets upon centrifugation in aqueous media.

Upon isolating the cell lysate and membrane-containing fractions of the cell culture system, it may be desirable to analyze the mixture in order to ascertain which component of the mixture contains the fusion protein of interest. This can be performed using standard molecular biology techniques known in the art, e.g., by SDS-PAGE analysis of the cell lysate and membrane-containing fractions of the expression system. A finding that the fusion protein of interest is contained primarily in the membrane-containing fractions may indicate that the fusion protein aggregates within inclusion bodies. In these cases, one of skill in the art may solubilize the inclusion bodies using, e.g., a chemical detergent, followed by treating the protein-containing fraction with a buffer that promotes re-folding of the fusion protein. Method for recovering proteins from inclusion bodies are described, e.g., in Francis et al. J. Mol. Endocrinol. 8:213-223 (1992), the disclosure of which is incorporated herein by reference.

Optionally, the fusion protein may be synthesized using chemical synthesis techniques, such as by solid phase peptide synthesis methods known in the art.

Following preparation, the fusion protein can then be loaded onto a cation-exchange chromatography column, e.g., a column that contains an agarose or a polystyrene matrix. The matrix may include one or more negatively-charged moieties, such as a strong cation exchanger (e.g., a sulfopropyl-containing molecule) or a weak cation exchanger (e.g., a carboxy-containing molecule). The column may optionally be washed with one or more buffers containing one or salts (e.g., sodium chloride) and buffer components (e.g., phosphate-buffered saline solutions known in the art or sodium borate). The buffers used to wash the column may be capable of removing one or more impurities from the column (e.g., contaminating polypeptides or polynucleotides) without disrupting the binding of the fusion protein to the negatively-charged moieties within the column. The fusion protein can subsequently be eluted from the column by treating the column with one or more buffers containing an elevated concentration of a cation (e.g., lithium (Li⁺), sodium (Na⁺), potassium (K⁺), ammonium (NH₄⁺), magnesium (Mg²⁺), calcium (Ca²⁺), or zinc (Zn⁺)). The concentration of the cation in the elution buffer may be increased gradually, e.g., by implementing a gradient elution profile in which the concentration of the cation increases linearly over a period of time, such as 30-60 minutes. The concentration of the cation in solution may also be increased abruptly, e.g., as described in Table 3 below. Alternatively, the isocratic elution techniques can be used in which the concentration of cation in solution remains at a constant level. The presence of the cation disrupts the binding of the matrix-binding domain of the fusion protein to the negatively-charged moieties within the column by competing with the fusion protein for binding sites on the matrix. As the elution buffer flows through the column, the fusion protein dissociates from the matrix and is recovered from the column. The fusion protein can be detected using standard detection methods known in the art, e.g., by analyzing the column eluate using UV-Vis spectroscopy and monitoring the absorbance of the eluate at about 276 nm, an absorbance signature characteristic of proteins containing aromatic residues.

The fusion protein can subsequently be analyzed using one or more analytical techniques known in the art, e.g., SDS-PAGE or liquid chromatography (e.g., size exclusion chromatography or reverse-phase high-pressure liquid chromatography) in order to ascertain the purity of the fusion protein. Optionally, the fusion protein can be further purified, e.g., by size-exclusion chromatography or hydrophobic interaction chromatography. Optionally, hydrophobic moieties (e.g., butyl, octyl, or phenyl chains) can be incorporated within the cation-exchange column so as to form a mixed-mode column. The fusion protein can then be eluted by treating the column with a buffer containing a lyotropic salt (e.g., ammonium sulfate, potassium phosphate, sodium acetate, sodium chloride, or potassium thiocyanate) in order to promote desorption of the fusion protein from the mixed-mode resin.

Exemplary methods that typify the general protocol described above are provided in Examples 1-4, below.

### Matrix-Binding Domains

Matrix-binding domains useful in conjunction with the methods of the invention include those that bind extracellular matrix glycosaminoglycans, such as heparin, heparan sulfate, chondroitin sulfate, dermatan sulfate, and hyaluronic acid, among others. Matrix-binding domains that can be fused to therapeutic polypeptides are described in detail, e.g., in WO 2014/004467, WO 2014/004465, and in US 2008/0138323, the disclosures of each of which are incorporated herein by reference. Non-limiting examples of matrix-binding domains that can be fused to a polypeptide (e.g., a therapeutic polypeptide) to form a fusion protein that can be purified by cation-exchange chromatography techniques described herein are provided in Table 1 below, as well as variants thereof, such as variants that have at least 85% sequence identity (e.g., at least 85%, 90%, 95%, 97%, 98%, 99%, or more, sequence identity) thereto.

TABLE 1
Exemplary matrix-binding domains

| SEQ | |
|---|---|
| ID | Amino acid |
| NO. | sequence of matrix-binding domain |
| 1 | KKKRKGKGLGKKRDPCLKKYKG |
| 2 | MKRKKKGKGLGKKRDPCLRKYK |
| 3 | KRKKKGKGLGKKRDPCLRKYK |
| 4 | MKRKKKGKGLGKKRDPSLRKYK |
| 5 | MKRKKKGKGLGKKRDPRLRKYK |
| 6 | MKRKKKGKGLGKKRDPKLRKYK |
| 7 | KRKKKGKGLGKKRDPSLRKYK |
| 8 | KRKKKGKGLGKKRDPRLRKYK |
| 9 | KRKKKGKGLGKKRDPKLRKYK |
| 10 | RIQNLLKITNLRIKFVK |
| 11 | RYVVLPRPVCFEKGTNYTVR |
| 12 | KQNCLSSRASFRGCVRNLRLSR |
| 13 | YKKIIKKL |
| 14 | CKNGGFFLRIHPDGRVDGVREK |
| 15 | YTSWYVALKRTGQYKLGSKTGPGQKAILFLP |
| 16 | AKLNCRLYRKANKSSKLVSANRLFGDK |
| 17 | LRKLRKRLLRDADDLQKRLAVYQ |
| 18 | PLQERAQAWGQERLRARMEEMGSRTRDRLDEVKEQVAERAKL |
| 19 | KGKMHKTCYF |
| 20 | KHKGRDVILKKDVR |
| 21 | KKHAEKNWFVGLKKNGSCKRGP |
| 22 | KGGRGTPGKPGPRGQRGPTGPRGERGPRGITGK |
| 23 | GEFYDLRLKGDK |
| 24 | HRHHPREMKKRVEDL |
| 25 | EKTLRKWLKMFKKR |
| 26 | RRRPKGRGKRRREKQRPTDCHL |
| 27 | QPTRRPRPGTGPGRRPRPRPRP |

### Therapeutic Polypeptides

Fusion proteins that can be purified according to the methods of the invention include those that contain a therapeutic polypeptide. In some embodiments, the fusion protein contains a matrix-binding domain at the N-terminus and a therapeutic polypeptide at the C-terminus of the fusion protein. In other embodiments, the fusion protein contains a therapeutic polypeptide at the N-terminus and a matrix-binding domain at the C-terminus. Exemplary therapeutic polypeptides are described, e.g., in WO 2014/004465, the disclosure of which is incorporated herein by reference. Such polypeptides include, without limitation, growth and differentiation factor 11 (GDF11), stromal cell-derived factor 1 (SDF-1), growth and differentiation factor 8 (GDF8), insulin-like growth factor 1 (IGF-1), parathyroid hormone (PTH), parathyroid hormone related peptide (PTHrP), interleukin 1 receptor antagonist (IL-1RA), fibroblast growth factor 9 (FGF-9), fibroblast growth factor 18 (FGF-18), high-mobility group protein 2 (HMG-2), hepatocyte growth factor, transforming growth factor β(TGFβ), transforming growth factor β3 (TGFβ3), bone morphogenetic protein 2 (BMP2), bone morphogenetic protein 7 (BMP7), angiopoietin-like 3 (ANGPTL3), and somatostatin (SST).

Additional therapeutic polypeptides that can be purified according to the methods of the invention include antibodies and antigen-binding fragments thereof. Exemplary antibodies for use with the methods of the invention include infliximab, adalimumab, etanercept, and an anti-nerve growth factor antibody.

Therapeutic polypeptides that can be purified according to the methods of the invention also include neurotrophins, such as nerve growth factor (NGF), brain-derived neurotrophic factor (BDNF), neurotrophin-3 (NT-3), and neurotrophin-4 (NT-4).

Additional therapeutic polypeptides that can be conjugated to matrix-binding domains to form fusion proteins and that may be purified according to the methods of the invention include neurotrophic factors. Exemplary neurotrophic factors include, without limitation, glial cell line-derived neurotrophic factor (GDNF), neurturin (NRTN), artemin (ARTN), persephin (PSPN), ciliary neurotrophic factor (CNTF), mesencephalic astrocyte-derived neurotrophic factor (MANF), and conserved dopamine neurotrophic factor (CDNF).

Other therapeutic polypeptides that can be purified according to the methods of the invention include cytokines, such as interleukin-4 (IL-4), interleukin-6 (IL-6), interleukin-10 (IL-10), interleukin-11 (IL-11), interleukin-27 (IL-27), leukemia inhibitory factor, cardiotrophin 1, neuropoietin, and cardiotrophin-like cytokine.

Therapeutic polypeptides that can be purified according to the methods of the invention also include neuroprotection agents, such as Neuregulin-1 and vascular endothelial growth factor (VEGF).

Additional examples of therapeutic polypeptides that can be purified according to the methods of the invention include variants of the above-described peptides that retain the biological activity of the original molecule. For instance, a variety of IGF-1 variants can be produced containing substitutions at one or more positions, such as those described in U.S. Pat. No. 8,759,299, the disclosure of which is incorporated herein by reference.

### Linkers

Fusion proteins that can be purified according to the methods of the invention include those that contain a matrix-binding domain covalently bound to a polypeptide (e.g., a therapeutic polypeptide as described herein). Optionally, these domains may be joined by a linker. For instance, a therapeutic polypeptide can be joined to a matrix-binding domain by forming a covalent bond between the therapeutic polypeptide and a linker. This linker can then be subsequently conjugated to a matrix-binding domain, or the linker can be conjugated to a matrix-binding domain prior to conjugation to the therapeutic polypeptide. Examples of linkers that can be used for the formation of a fusion protein include polypeptide linkers, such as those that contain naturally occurring or non-naturally occurring amino acids. Exemplary polypeptide linkers include those that contain hydrophilic substituents, such as hydroxyl moieties, so as to promote the solubility of the fusion protein in aqueous solution. For instance, a linker may contain glycine (Gly) and/or serine (Ser) residues, e.g., according to the formula [(Gly)ₐ(Ser)_{b}]_{c}, wherein a, b, and c are independently integers from 0 to 20. For instance, a linker useful with the methods of the invention may be characterized by the above formula, wherein a=3, b=1, and c is an integer from 1 to 6 (e.g., 1, 2, 3, 4, 5, or 6). In some embodiments, the linker may be characterized by the above formula, wherein a=4, b=1, and c is an integer from 1 to 6 (e.g., 1, 2, 3, 4, 5, or 6). In certain cases, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those known in the art, or through recombinant expression of a polynucleotide encoding the fusion protein in a cell. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage, among other techniques as described, e.g., in Leriche, et al. Bioorg. Med. Chem. 20:571-582 (2012), the disclosure of which is incorporated herein by reference.

Fusion proteins containing matrix-binding domains may also be produced using, e.g., a linker that joins the matrix-binding domain to a therapeutic polypeptide and that is cleavable by naturally-occurring enzymes. Examples of such linkers include polypeptides that include an amino acid sequence that is selectively recognized and cleaved by proteases, such as, e.g., trypsin, chymotrypsin, thrombin, and pepsin, among others.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. R&D Scale Purification of MB-URA from 2 L Growth with Scalable Chromatography Steps

An early step in developing therapeutics is to accurately determine their pharmacological activity. For protein therapies, it can be a challenge to produce protein that is pure enough to test at research & development scales of production without introducing modifications to the sequence that could potentially affect their activity.

Initial attempts to produce MB-IL1RA for testing of activity were unsuccessful because the fusion tags which were used to allow purification and identification of the proteins interfered with the activities of both URA and MB-IL1RA. Specifically, a tag containing a hexahistidine sequence was used to allow purification by Ni-NTA affinity media of IL-1RA and MB-IL1RA. A cell-based NF-kB-induced luciferase assay was used to test activity of the proteins. The assay demonstrated greatly reduced activity of both the 6×His-MB-IL1RA and the 6×His-IL1RA compared to KINERET® (anakinra, untagged pharmaceutical-grade Met-IL1RA, Sobi, Inc., Waltham, Mass.). We were therefore unable to determine the activity of the MB-IL1RA protein relative to URA.

We subsequently tested the idea that the proteins could be purified at the research scale using the properties of the MB peptide without any additional affinity tags.

Three untagged MB-IL1RA expression constructs were generated in pET29(a) expression vectors (Genscript USA):

1. MB-IL1RA (sequence depicted below)

### 2. MB-IL1RA C17K

### 3. MB-IL1RA C17R

The MB-IL1RA protein sequence was as follows (underlined residues represent the matrix-binding domain; bolded residues denote the linker between the matrix-binding domain and the IL-1RA peptide):

(SEQ ID NO: 28)
MKRKKKGKGL GKKRDPCLRK YKGGGSRPSG RKSSKMQAFR
IWDVNQKTFY LRNNQLVAGY LQGPNVNLEE KIDVVPIEPH
ALFLGIHGGK MCLSCVKSGD ETRLQLEAVN ITDLSENRKQ
DKRFAFIRSD SGPTTSFESA ACPGWFLCTA MEADQPVSLT
NMPDEGVMVT KFYFQEDE

The remaining two MB-IL1RA proteins contained single amino-acid substitutions at the cysteine in amino acid position 17 (C17R and C17K) for improvement of matrix binding strength.

Plasmid DNA from each of the above listed constructs was transformed into T7 Express E. coli competent BL21(DE3) cells. Transformed plasmids were grown overnight at 37° C. in Luria-Bertani (LB) medium with kanamycin. 1.2 mL of overnight cell growth was diluted in 10 mL medium and grown to an OD_{600 nm} reading of 1.0 and then induced at 32° C. with 1 mM IPTG for five hours.

A portion of un-induced medium was reserved for analysis. Following induction, cells were pelleted, decanted and the pellet was frozen. Cell pellets were re-suspended in lysis buffer, lysed by sonication and clarified by centrifugation. The liquid phase of this centrifugation product was taken as the soluble fraction and the pellet was re-suspended in lysis buffer as the insoluble fraction. Samples of each condition were run under reducing conditions and resolved by SDS PAGE gel.

### Purification

A cation-exchange chromatography column (HiTrap SP Sepharose FF, GE Healthcare) was equilibrated with 5 column volumes of Buffer A1 (25 mM HEPES pH 7.4, 400 mM NaCl, 2 mM 3-ME). Cells were re-suspended in Lysis buffer (4 mL/gram protein of 25 mM HEPES pH 7.4, 400 mM NaCl, 2 mM β-ME, protease inhibitors, benzonase, 0.05% Triton X-114), and lysed by pipetting and microfluidizer. The lysate was cleared by centrifugation at 20,000 g for 30 min. The supernatant (35 mL) was then loaded on the 1 mL column and washed with 10 column volumes Buffer A1, then 50 column volumes Buffer A1 with 0.1% Trition X-114, and then 10 column volumes of Buffer A1. The desired protein was eluted by 100 column volumes of buffer A1 with a 400 mM to 1M NaCl gradient. 1 mL fractions were collected and the protein concentration was measured by Bradford assay.

### Size-Exclusion Chromatography

As an additional purification step, size-exclusion chromatography was performed. Fractions 25-90 were pooled, a total of 60 mL at a protein concentration of 0.8 mg/mL. The pooled fractions were concentrated with 10,000 MW-cut off centrifugal concentrators and sterilized by filtration through a 0.2 μm pore size filter, yielding 5 mL at a concentration of 8.36 mg/mL. The sample was then loaded on to a Superdex 26/60 size-exclusion column pre-equilibrated in 25 mM HEPES, pH 7.4, 150 mM NaCl, 2 mM β-ME, and 5% glycerol.

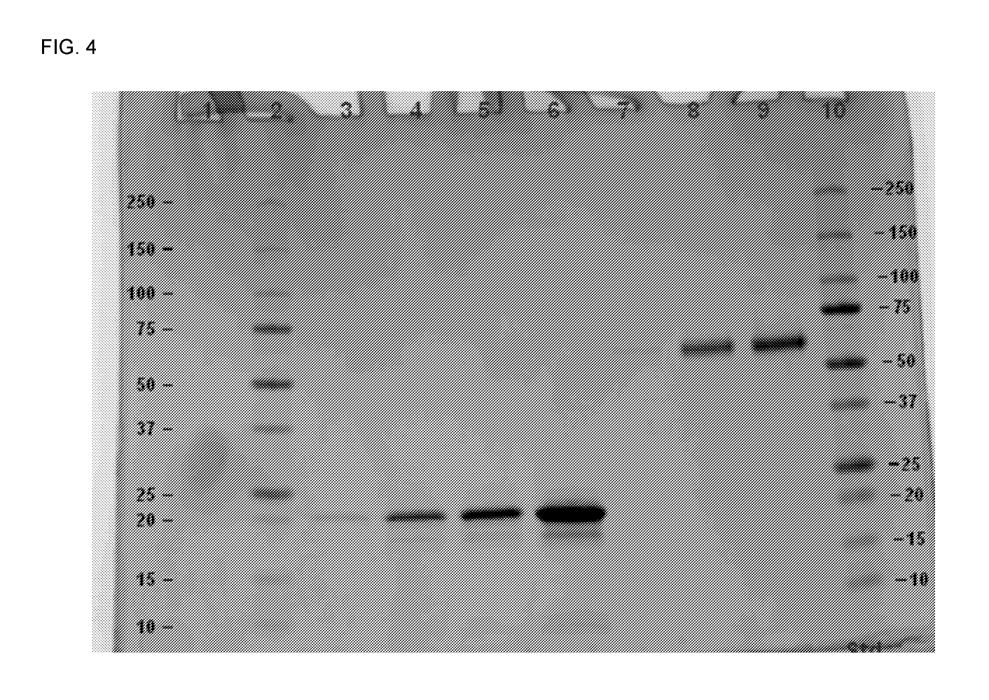

Fractions A86-A95 were subsequently pooled and analyzed by SDS-PAGE to verify purity (FIG. 4). Final yield after purification was 18 mL of protein at a concentration of 1.15 mg/ml, as measured by Bradford assay, for a total of 20 mg purified protein from 2 L bacterial cell growth (10 mg purified protein/L bacterial culture).

To verify purity from E. coli host cell contaminants, endotoxin levels were measured by an LAL Chromogenic Endotoxin Quantitation Kit (Pierce). The mean endotoxin reading was 0.23 EU/μg, low enough to qualify for use in experimental research work.

To verify identity, the protein was then analyzed for exact molecular weight by MALDI-TOF mass spectrometry. Compared to the theoretical molecular weight of MB-IL1RA of 20,013.9 Daltons, the measured mass spectrometry result was 20,001 Da, a difference of 0.6%, confirming identity.

### Example 2. Commercially Scalable Production and Purification of MB-URA from 15 L Bacterial Fermenter

### MB-URA Sequence

The amino acid sequence was as follows (single letter code):

(SEQ ID NO: 29)
MKRKKKGKGLGKKRDPRLRKYKGGGSRPSGRKSSKMQAFRIWDVNQKTFY
LRNNQLVAGYLQGPNVNLEEKIDVVPIEPHALFLGIHGGKMCLSCVKSGD
ETRLQLEAVNITDLSENRKQDKRFAFIRSDSGPTTSFESAACPGWFLCTA
MEADQPVSLTNMPDEGVMVTKFYFQEDE

### 15-L Fermentation

Fermentations were carried out in 15-L (Total volume) Biolafitte stainless-steel vessels with the impeller configuration of one Rushton and one marine-style down-pumper. Batch medium consisting of 0.55% potassium phosphate monobasic, 0.24% magnesium sulfate heptahydrate, 0.64% glycerol, 2.75% yeast extract, and 0.05% antifoam, pH adjusted to 7.0 with 15% v/v ammonium hydroxide, was added to the vessel to a volume of 5.5 L. The vessels were then sterilized-in-place (SIP) with steam. Trace metals solution (0.0059% v/v sulfuric acid, 0.0067% w/v iron (II) sulfate heptahydrate, 0.0007% w/v manganese sulfate monohydrate, 0.0008% w/v zinc sulfate heptahydrate, 0.17% w/v copper sulfate pentahydrate, 0.02% w/v calcium chloride dihydrate), kanamycin sulfate (50 μg/mL), and 0.19% w/v citric acid solution are added as a post-sterile additions via 0.22 μm filter. The fermenters were inoculated at a 6% volume/volume ratio, or 420 mL of culture added to each 7 L batch. Cultivation was performed in fed-batch mode with the following conditions: temperature set at 32° C.; one-sided pH control at 7.0 with 15% v/v ammonium hydroxide solution; and dissolved oxygen at 20%, maintained by cascading agitation and then blending in pure 02 with the air to maintain a total gas flow rate of 12 L/min. Once the OD₆₀₀ of the fermenter reached 60 AU/cm, expression was induced by adding IPTG to a final concentration of 233 μM. Cultures were induced for four hours or longer, with hourly sampling.

### Cell Harvest, Lysis, and Acid Clarification

Once the culture was induced, the whole cells were harvested by aliquoting into 1-L high-speed centrifuge bottles and centrifuging at 8000 rpm (14,000×g) for 20 minutes. All whole cell broth was stored at 2-8° C. during centrifugation. The supernatant was removed and the cell pellet was stored at −20° C. Frozen cells were allowed to thaw and were re-suspended in lysis/wash buffer (50 mM Tris, 5 mM EDTA, pH 7.7 at 4° C.) at a 1:6.7 ratio of cell mass-to-suspension mass (approximately 15% solids). Re-suspended cells were passed through a Niro homogenizer three times to ensure complete lysis and DNA shearing. Cell debris was removed by centrifugation, before host cell proteins were precipitated by acidifying the lysate to pH 5. The lysate was cleared by centrifugation and filtered.

### Cation-Exchange Chromatography

Cation-exchange chromatography (CEX) was used for the capture and initial purification of MB-URA from the acidified, clarified lysate. Column setup conditions are shown in Table 2. The pre-equilibrated column was loaded with 10 mL of neutralized (pH 7) MB-IL1RA solution. The loading solution was pumped onto the column via a syringe drive at a flow rate of 1.25 mL/min. After loading was completed, the column was connected with a Waters Alliance HPLC system, and washed with 4 column volumes (CV) of Buffer A, also at 1.25 mL/min. The column was then eluted using the gradient shown in Table 3. During the gradient, absorbance was monitored at 276 nm using a Waters 2996 photodiode array detector. Eluent collected near the center of the main peak retention time range (40-42 min) was pooled for analytical characterization and bioassay.

TABLE 2
CEX Column Conditions

| | |
|---|---|
| Column packing: | SP Sepharose FF (GE) |
| Column volume: | 5 mL |
| Flow rate: | 1.25 mL/min |
| Buffer A: | 50 mM NaCl, 50 mM Borate (Na) pH 9.6 |
| Buffer B: | 2M NaCl, 50 mM Borate (Na) pH 9.6 |
| Temperature: | ambient |
| Pre-equilibration: | 10 column volumes Buffer A |

TABLE 3
CEX Gradient

| Time (min) | Flow Rate (mL/min) | % A | % B |
|---|---|---|---|
| initial | 1.25 | 100 | 0 |
| 10 | 1.25 | 100 | 0 |
| 50 | 1.25 | 50 | 50 |
| 58 | 1.25 | 50 | 50 |
| 66 | 1.25 | 100 | 0 |
| 70 | 1.25 | 100 | 0 |

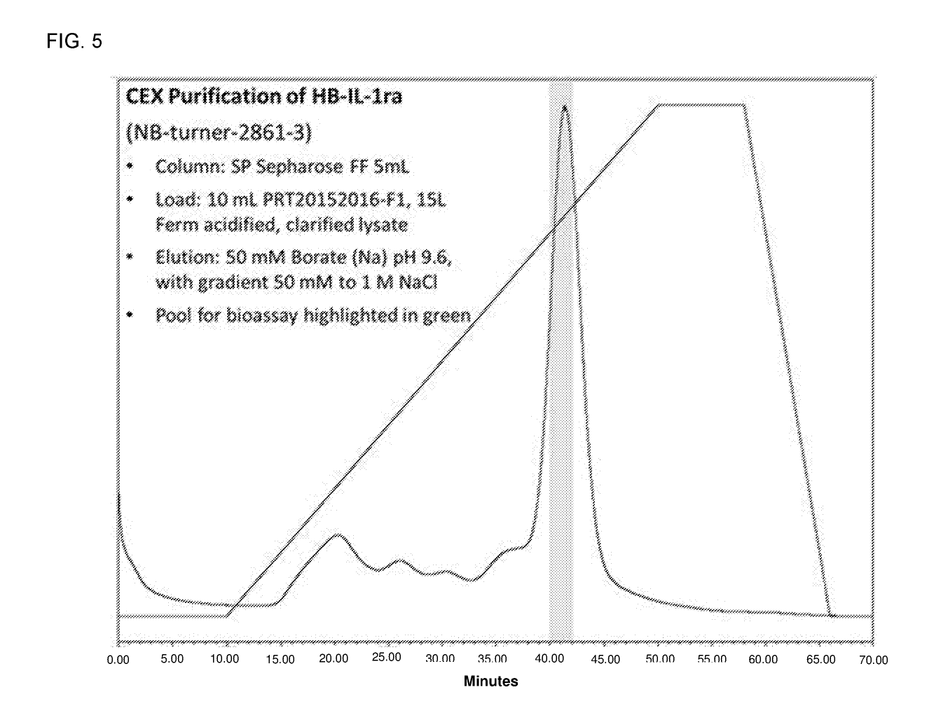

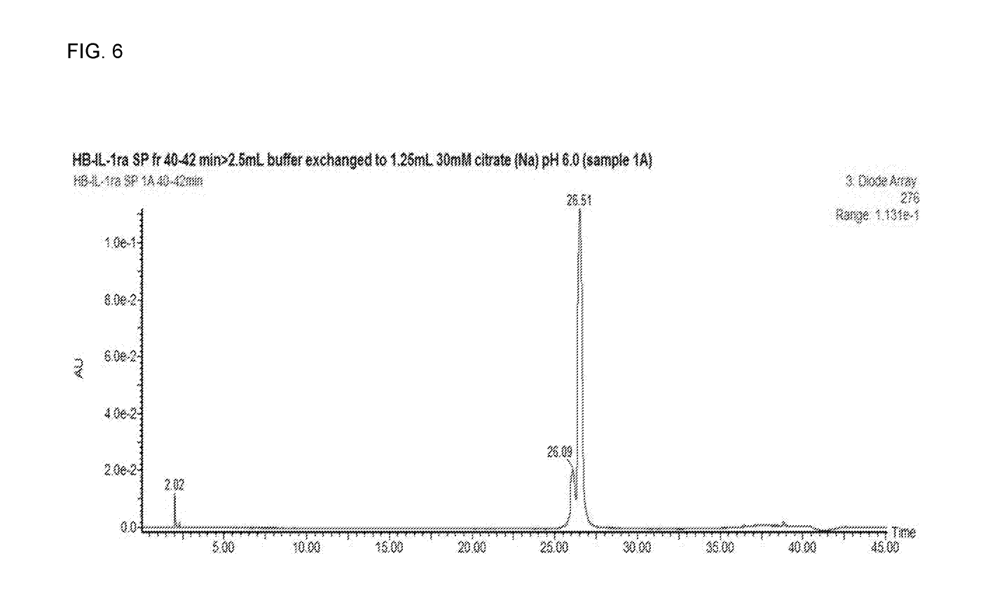

The UV (276 nm) absorbance trace obtained during the CEX capture and purification is shown in FIG. 5, which also shows the retention time range of eluent collected. The CEX eluent was concentrated two-fold and buffer exchanged into 30 mM citrate (sodium), pH 6.0. The absorbance traces obtained with CEX purified MB-IL1RA exhibits a major peak (FIG. 6) which is ˜95% pure, based on the high resolution mass spectrometry data. A small peak eluting earlier than the main peak with molecular weights lower by approximately 2 Da are also observed with the sample. The mass differences observed for the molecular weights of the protein eluting in these small peaks in comparison to those obtained for the major species are consistent with proteins having the same amino acid sequence, but containing a single disulfide. The endotoxin levels in the material collected were determined to be 5.8 EU/mL, low enough for research and development stage activities.

### MB-IL1RA Fusion Activity

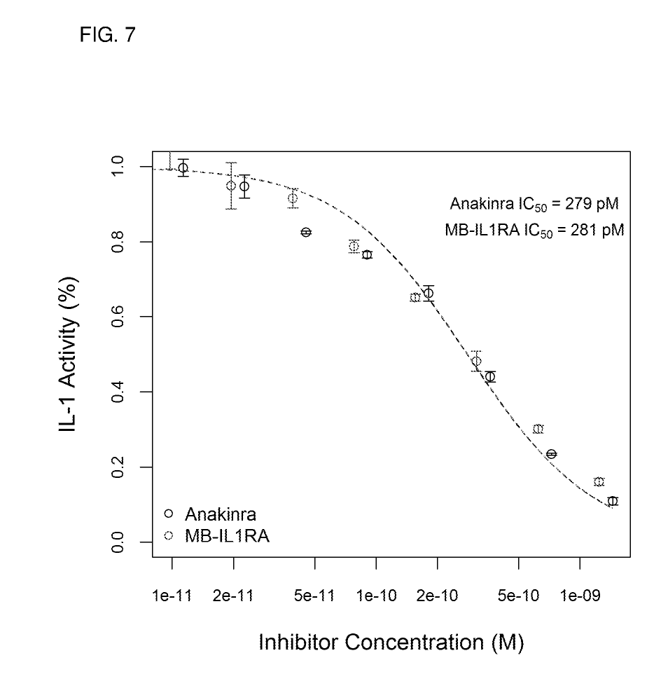

Beyond the addition of an amino-terminal methionine, it has been very challenging to modify IL-1RA by fusion at either end of the molecule without substantial loss in activity (Shamji et al. Arthritis Rheum. 56:3650-3661 (2007)). We therefore tested the IL-1-inhibitory activity of the MB-IL1RA fusion protein purified by cation-exchange chromatography in an NFkB response element-driven luciferase reporter cell assay, which confirmed no loss of activity for MB-IL1RA compared to anakinra (FIG. 7).

### Example 3. Purification of MB-IGF-1 Fusion Protein by Cation-Exchange Chromatography

Using the methods of the invention, one of skill in the art can purify a fusion protein containing IGF-1 bound to a matrix-binding domain, such as a matrix-binding domain having the amino acid sequence of any one of SEQ ID NOs: 1-27. For instance, a fusion protein containing the matrix-binding domain of SEQ ID NO: 4 and the amino acid sequence of IGF-1, or a fragment thereof that retains the biological activity of IGF-1 (e.g., the ability to bind the endogenous IGF-1 receptor and potentiate cell proliferation and synthesis of matrix proteoglycans). An exemplary human IGF-1 sequence is shown below:

(SEQ ID NO: 30)
GPETLCGAELVDALQFVCGDRGFYFNKPTGYGSSSRRAPQTGIVDECCFR
SCDLRRLEMYCAPLKPAKSARSVRAQRHTDMPKTQKEVHLKNASRGSA

One of skill in the art can express the IGF-1 peptide of SEQ ID NO: 30 as a fusion protein containing the matrix-binding domain of SEQ ID NO: 4, e.g., wherein the matrix-binding domain is located at the N-terminus or at the C-terminus of the resulting fusion protein. The protein may optionally contain a linker, such as glycine/serine-containing linker described herein, positioned between these two peptides. This fusion protein can be expressed using cell-based expression techniques, such as by inducing the synthesis of the fusion protein by treating a bacterial cell (e.g., an E. coli cell) containing a vector in which a gene encoding the fusion protein is under the control of a T7 promoter with isopropyl-β-D-thiogalactoside (IPTG) so as to promote the expression of T7 RNA polymerase. This induction process may desirably be performed once the bacterial cells containing this vector have reached an optimal cell density in culture, e.g., once the cells have been cultured so as to exhibit an OD₆₀₀ of from about 0.4 to about 0.8 as measured using conventional spectrophotometric techniques known in the art.

The resulting fusion protein can subsequently be prepared for cation-exchange chromatography by lysing the bacterial cells, e.g., as described herein, extracting the fusion protein from inclusion bodies, and dissolving the fusion protein in one or more buffers that promote the re-folding of the fusion protein, e.g., such that the IGF-1 peptide exhibits a spatial conformation similar to that of endogenous human IGF-1. Re-folding of the fusion protein can be monitored, e.g., using circular dichroism (CD) techniques known in the art. Methods for the extraction of proteins from inclusion bodies are described, e.g., in Francis et al. J. Mol. Endocrinol. 8:213-223 (1992), the disclosure of which is incorporated herein by reference.

Upon re-folding, the fusion protein can optionally be dissolved in a suitable buffer, such as a buffer containing a salt (e.g., NaCl) and one or more protease inhibitors at a pH of from about 7.0 to about 8.5. The mixture containing the fusion protein can then be loaded onto a cation-exchange column, e.g., a Sepharose column containing anionic sulfopropyl moieties covalently bound to the agarose resin. The purification may be performed on a small scale, e.g., by manually eluting the fusion protein from the column by treating the column with specified quantities of eluent containing a high concentration (e.g., 1 M or greater) of NaCl. Alternatively, the purification may be performed on a larger scale by placing the column in fluid communication with one or more pumps, such as those used for traditional high pressure liquid chromatography (HPLC) techniques known in the art. The pumps can be used to direct eluent containing an elevated concentration of NaCl relative to the buffer used to re-suspend the bacterial cell lysate through the column containing the anionic resin. Optionally, an isocratic elution program can be used by exposing the column to solutions containing discrete NaCl concentrations, e.g., as described in Table 3, above. A continuous gradient elution pattern may also be used, wherein the concentration of NaCl in the elution buffer is linearly increased over a period of time (e.g., 30-60 minutes). The separation can be monitored electronically, e.g., by analyzing the column eluate using a UV-Vis detector. Using this technique, protein-containing fractions can be identified by monitoring the absorbance of the eluate at about 280 nm, an absorbance that is characteristic of samples containing aromatic side chain functionality (e.g., tyrosine and tryptophan). The fractions collected from the ion-exchange chromatography can subsequently be analyzed using conventional SDS-PAGE techniques to verify purity of the MB-IGF-1 fusion protein.

### Example 4. Purification of MB-URA Fusion Protein by Mixed Mode Chromatography

MB-URA, e.g., containing the amino acid sequence of SEQ ID NO: 28 or SEQ ID NO: 29, can be produced using recombinant protein expression techniques described herein or known in the art, such as by the transformation of a bacterial cell with a vector containing a gene encoding the MB-IL1RA fusion protein under the control of an inducible promoter. The mixture containing the fusion protein can then be loaded onto a mixed-mode column containing anionic (e.g., sulfopropyl) moieties as well as hydrophobic molecules (e.g., molecules containing unsaturated aliphatic side chains, such as n-octyl groups, or aromatic molecules that are electrostatically neutral within a pH range of from about 7 to about 10, such as pyridine-containing molecules) covalently bound to the agarose resin. The purification may be performed on a small scale, e.g., by manually eluting the fusion protein from the column by treating the column with specified quantities of eluent containing a high concentration (e.g., 1 M or greater) of NaCl. Alternatively, the purification may be performed on a larger scale by placing the column in fluid communication with one or more pumps, such as those used for traditional high pressure liquid chromatography (HPLC) techniques known in the art. The pumps can be used to direct eluent containing an elevated concentration of NaCl relative to the buffer used to re-suspend the bacterial cell lysate through the column containing the anionic resin. Optionally, the pH of the elution buffer may be gradually decreased as the separation continues, e.g., so as to induce protonation of the pyridine nitrogen and weaken the interaction between the matrix-binding domain and the increasingly cationic pyridinium moieties bound to the resin. The separation can be monitored electronically, e.g., by analyzing the column eluate using a UV-Vis detector. Using this technique, protein-containing fractions can be identified by monitoring the absorbance of the eluate at about 280 nm, an absorbance that is characteristic of samples containing aromatic side chain functionality (e.g., tyrosine and tryptophan). The fractions collected from the ion-exchange chromatography can subsequently be analyzed using conventional SDS-PAGE techniques to verify purity of the MB-IL1RA fusion protein.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of purifying a fusion protein comprising a matrix-binding domain, said method comprising contacting a mixture of polypeptides comprising the fusion protein with a substance comprising one or more negatively-charged agents so that the matrix-binding domain of the fusion protein specifically binds said one or more negatively-charged agents from said mixture, thereby producing a mixture that is enriched with said fusion protein.

2. The method of claim 1, wherein said substance comprising one or more negatively-charged agents is contained within a column, optionally wherein said column is in fluid connection with one or more pumps.

3. (canceled)

4. The method of claim 1, wherein said matrix-binding domain is capable of specifically binding a glycosaminoglycan selected from the group consisting of heparin, heparan sulfate, chondroitin sulfate, dermatan sulfate, and hyaluronic acid.

5. The method of claim 1, wherein said matrix-binding domain has at least 85% sequence identity to, or the amino acid sequence of, any one of SEQ ID NOs: 1-27.

6. The method of claim 1, wherein said fusion protein comprises a therapeutic polypeptide, optionally wherein said therapeutic polypeptide:
(a) is selected from the group consisting of growth and differentiation factor 11 (GDF11), stromal cell-derived factor 1 (SDF-1), growth and differentiation factor 8 (GDF8), insulin-like growth factor 1 (IGF-1), parathyroid hormone (PTH), parathyroid hormone related peptide (PTHrP), interleukin 1 receptor antagonist (IL-1RA), fibroblast growth factor 9 (FGF-9), fibroblast growth factor 18 (FGF-18), high-mobility group protein 2 (HMG-2), hepatocyte growth factor, transforming growth factor β (TGFβ), transforming growth factor β3 (TGFβ3), bone morphogenetic protein 2 (BMP2), bone morphogenetic protein 7 (BMP7), angiopoietin-like 3 (ANGPTL3), and somatostatin (SST);
(b) comprises an antibody or an antigen-binding fragment thereof, optionally wherein said antibody is selected from the group consisting of infliximab, adalimumab, etanercept, and an anti-nerve growth factor antibody;
(c) is a neurotrophin, optionally wherein said neurotrophin is selected from the group consisting of nerve growth factor (NGF), brain-derived neurotrophic factor (BDNF), neurotrophin-3 (NT-3), and neurotrophin-4 (NT-4);
(d) is a neurotrophic factor, optionally wherein said neurotrophic factor is selected from the group consisting of glial cell line-derived neurotrophic factor (GDNF), neurturin (NRTN), artemin (ARTN), persephin (PSPN), ciliary neurotrophic factor (CNTF), mesencephalic astrocyte-derived neurotrophic factor (MANF), and conserved dopamine neurotrophic factor (CDNF);
(e) is a cytokine, optionally wherein said cytokine is selected from the group consisting of interleukin-4, interleukin-6, interleukin-10, interleukin-11, interleukin-27, leukemia inhibitory factor, cardiotrophin 1, neuropoietin, and cardiotrophin-like cytokine; or
(f) is a neuroprotection agent, optionally wherein said neuroprotection agent is selected from the group consisting of Neuregulin-1 and vascular endothelial growth factor (VEGF).

7-17. (canceled)

18. The method of claim 1, wherein said fusion protein comprises a linker, optionally wherein said linker:
(a) comprises a peptide linker comprising one or more amino acids, such as D- or L-amino acids and non-naturally occurring amino acids, or combinations thereof, or a non-peptide linker; and/or
(b) is cleavable, optionally wherein said linker is cleavable by a process selected from the group consisting of enzymatic hydrolysis, photolysis, hydrolysis under acidic conditions, hydrolysis under basic conditions, oxidation, disulfide reduction, nucleophilic cleavage, and organometallic cleavage.

19-21. (canceled)

22. The method of claim 18, wherein the linker comprises a polypeptide of the formula [(Gly)ₐ(Ser)_{b}]_{c}, wherein a, b, and c are independently integers from 0 to 20, optionally wherein:
(a) b is 0, optionally wherein a is 3; or
(b) a is 3 or 4, and b is 1, optionally wherein c is an integer from 1 to 6.

23-26. (canceled)

27. The method of claim 1, wherein said fusion protein is isolated from a cell, optionally wherein:
(a) said cell is a eukaryotic cell, optionally wherein said eukaryotic cell is a mammalian cell; or
(b) said cell is a prokaryotic cell, optionally wherein said prokaryotic cell is a bacterial cell, optionally wherein said bacterial cell is an E. coli cell, optionally wherein said fusion protein is produced by treating said E. coli cell with isopropyl-β-D-thiogalactoside (IPTG).

28-33. (canceled)

34. The method of claim 1, wherein said method comprises contacting said one or more negatively-charged agents with a solution comprising a dissolved cation, optionally wherein:
(a) the contacting of said one or more negatively-charged agents with said solution comprising a dissolved cation causes said fusion protein to dissociate from said substance comprising one or more negatively-charged agents; and/or
(b) said dissolved cation is selected from the group consisting of lithium (Li⁺), sodium (Na⁺), potassium (K⁺), ammonium (NH₄⁺), magnesium (Mg²⁺), calcium (Ca²⁺), and zinc (Zn⁺).

35-36. (canceled)

37. The method of claim 34, wherein said method comprises contacting said one or more negatively-charged agents with a first solution comprising said dissolved cation, and subsequently contacting said one or more negatively-charged agents with a second solution comprising said dissolved cation, wherein the concentration of said dissolved cation in the second solution is greater than the concentration of said dissolved cation in said first solution, optionally wherein said method further comprises subsequently contacting said one or more negatively-charged agents with a third solution comprising said dissolved cation, wherein the concentration of said dissolved cation in the third solution is greater than the concentration of said dissolved cation in said first solution and said second solution.

38. (canceled)

39. The method of claim 37, wherein:
(a) the concentration of said dissolved cation in the first solution is from about 1 mM to about 100 mM, optionally wherein the concentration of said dissolved cation in the first solution is about 50 mM; and/or
(b) the concentration of said dissolved cation in the second solution is from about 500 mM to about 1.5 M, optionally wherein the concentration of said dissolved cation in the second solution is about 1 M; and/or
(c) the concentration of said dissolved cation in the third solution is from about 1.6 M to about 2.5 M, optionally wherein the concentration of said dissolved cation in the third solution is about 2 M; and/or
(d) said first, second, and third solutions flow through said substance comprising one or more negatively-charged agents at a rate of from about 1 to about 3 mL/minute, optionally wherein said first, second, and third solutions flow through said substance comprising one or more negatively-charged agents at a rate of about 1.25 mL/minute.

40-46. (canceled)

47. The method of claim 1, wherein:
(a) said one or more negatively-charged agents are selected from the group consisting of methanesulfonic acid, ethanesulfonic acid, propanesulfonic acid, benzenesulfonic acid, and acetic acid; and/or
(b) said one or more negatively-charged agents are covalently bound to said substance, optionally wherein said substance is a polysaccharide or polystyrene, optionally wherein said polysaccharide is agarose; and/or
(c) said substance additionally comprises one or more hydrophobic molecules.

48-52. (canceled)

53. The method of claim 1, said method further comprising:
a) (i) contacting the mixture that is enriched with said fusion protein with a material comprising a plurality of particles; and
(ii) separating polypeptides that flow through said material from polypeptides that remain within said material, and/or
b) (i) contacting the mixture that is enriched with said fusion protein with a material comprising one or more hydrophobic molecules; and
(ii) separating polypeptides that bind said one or more hydrophobic molecules from the mixture that is enriched with said fusion protein;
optionally wherein the average diameter of said plurality of particles is from about 1 μm to about 100 μm, optionally wherein the average diameter of said plurality of particles is from about 10 μm to about 50 μm, optionally wherein the average diameter of said plurality of particles is about 34 μm.

54-57. (canceled)

58. The method of claim 1, wherein said fusion protein comprises the amino acid sequence of SEQ ID NO: 28 or SEQ ID NO: 29.

59-119. (canceled)
